(19) United States
(12) Patent Application Publication
(10) Pub. No.: US 2012/0301457 A1
(43) Pub. Date: Nov. 29, 2012

(54) LIPID COFACTORS FOR FACILITATING PROPOGATION OF PRPsc

(76) Inventors: Surachai Supattapone, Hanover, NH (US); Nathan R. Deleault, Canaan, NH (US)

(21) Appl. No.: 13/522,724

(22) PCT Filed: Jan. 19, 2011
(86) PCT No.: PCT/US11/21683

§ 371 Date: Jul. 23, 2012

Related U.S. Application Data
(60) Provisional application No. 61/297,419, filed on Jan. 22, 2010.

(51) Int. Cl. G01N 33/577; C07K 2/00; A61K 38/02; A01N 37/46; C07H 99/00; A61K 31/70; C07H 21/00; A61K 48/00; A01N 57/16; A61K 33/00; A01N 59/00; A61K 35/66; C40B 30/00; A61P 31/00; A61K 35/00; C07K 1/00

(52) U.S. Cl. 424/115; 530/395; 435/7.1; 530/350; 514/1.1; 536/1.11; 514/23; 536/23.1; 514/44 R; 424/600; 424/780; 506/7

(57) ABSTRACT

The present invention embraces methods and kits for facilitating the propogation of PrP^{Sc}, and use of the same in increasing the sensitivity diagnostic assays and in identifying compounds that modulate the conversion of PrP^{c} to PrP^{Sc}.

### INTRODUCTION

This application claims benefit of priority to U.S. Provisional Application Ser. No. 61/297,419, filed Jan. 22, 2010, the content of which is incorporated herein by reference in its entirety.

The research underlying this invention was supported in part with funds from National Institutes of Health Grant Nos. NSO55875 and NSO46478. The United States Government has certain rights in this invention.

### BACKGROUND OF THE INVENTION

Infectious agents of prion diseases, such as Creutzfeldt Jakob Disease (CJD), are devoid of nucleic acid and instead are composed of a specific infectious protein (Prusiner (1982) Science 216:136-44). This protein, PrP^{Sc}, appears to be generated by the template-induced conformational change of a normally expressed neuronal glycoprotein, PrP^{C}, during the course of disease (Prusiner, S. B. (ed.) Prion Biology and Diseases, Cold Spring Harbor Laboratory Press, Cold Spring Harbor, N.Y. (1999). While numerous studies have established the conversion of PrP^{C} to PrP^{Sc} as the central pathogenic event of prion disease, cellular factors other than PrP^{C} which may be involved in the efficient catalysis of PrP^{Sc} are under investigation (Aguzzi & Weissmann (1997) Nature 389:795-8).

Various methods have been developed to enhance the amplification of PrP^{Sc} to increase the sensitivity of detecting PrP^{Sc}. Saborio, et al. ((2001) Nature 411:810-3) disclose the use of a protein misfolding cyclic amplification (PMCA) method wherein prion-infected tissue homogenates containing PrP^{C} are combined with normal brain homogenates in the presence of TRITON® X-100 and sodium dodecyl sulfate and subjected to repeated cycles of incubation and sonication to convert PrP^{C} in normal tissue to PrP^{Sc}. Lucassen, et al. ((2003) Biochemistry 42:4127-35) disclose a modified version of the PMCA method wherein the normal and prion-infected tissue homogenates are incubated under non-denaturing conditions for the conversion of PrP^{C} in normal tissue to PrP^{Sc}. Further, purified proteins and cell-lysate systems have been used to convert PrP^{C} to PrP (Kocisko, et al. (2000) Curr. Issues Mol. Biol. 2(3):95-101; Horiuchi & Caughey (1999) Structure Fold Des. 7:R231-R240; Saborio, et al. (1999) Biochem. Biophys. Res. Commun. 258:470-475). Optimal non-denaturing, cell-free conditions (KCl, MgCl₂, citrate buffer and sarkosyl) for the conversion of PrP^{C} to PrP^{Sc} have also been described (Horiuchi & Caughey (1999) EMBO J. 18:3193-3203). Cordeiro, et al. ((2001) J. Biol. Chem. 276:49400-9) teach that sequence-specific DNA binding to recombinant murine prion protein converts it from PrP^{C} to a non-infectious fibrillar state. Further, Nandi, et al. ((2002) Biochemistry 41:11017-11024) teach that the interaction between PrP^{C} and anions (sulfate/phosphate) in polyionic ligands such as sulfated glycosaminoglycan and DNA, induce unfolding of the prion protein and conversion to a non-infectious fibrillar prion protein. DebBurman, et al. ((1997) Proc. Natl. Acad. Sci. USA 94(25):13938-43) demonstrate that GroEL and Hsp104 (heat shock protein 104), significantly, but distinctly affect conversion of PrP^{C} to PrP^{Sc}.

Similarly, nucleic acids have been shown to bind to and promote the conformational change of recombinant PrP (Derrington, et al. (2002) CR Acad. Sci. III 325:17-23; Moscardini, et al. (2002) J. Mol. Biol. 318:149-59; Gabus, et al. (2001) J. Biol. Chem. 276:19301-9; Gabus, et al. (2001) J. Mol. Biol. 307:1011-21; Proske, et al. (2002) Chembiochem. 3:717-25; Weiss, et al. (1997) J. Virol. 71:8790-7; Zeiler, et al. (2003) Biotechnol. Appl. Biochem. 37:173-82; Nandi, et al. (2002) J. Mol. Biol. 322:153-61; Brimacombe, et al. (1999) Biochem. J. 342:605-613). Sulfated glycans and elevated temperature stimulate PrP(Sc)-dependent cell-free formation of protease-resistant prion protein. (Wong, et al. (2001) EMBO J. 20(3):377-86) as do RNA molecules (Deleault, et al. (2003) Nature 425(6959):717-20).

Purified PrP^{C} also converts into protease-resistant PrP^{Sc} in vitro in the absence of cellular cofactors (Kocisko, et al. (1995) Nature 370:471-4) and, thus, the PrP molecules themselves can drive species- and strain-specific PrP^{Sc} formation in vitro (Bessen, et al. (1995) Nature 375:698-700; Kocisko, et al. (1995) Proc. Natl. Acad. Sci. USA 92:3923-7). However, a 50-fold molar excess of purified PrP^{Sc} is required to drive conversion of purified PrP^{C}, suggesting that optimal efficiency of amplification may depend on the presence of cellular factors other than PrP^{C} (Caughey, et al. (1999) Methods Enzymol. 309:122-33). Transgenic experiments in mice and cultured cells also suggest that prion formation requires a catalytic factor “X” that has high affinity for positively charged residues at the C- and N-termini of PrP (Telling, et al. (1995) Cell 83:79-90; Kanecko, et al. (1997) Proc. Natl. Acad. Sci. USA 94:10069-74; Zulianello, et al. (2000) J. Virol. 74:4351-60; Perrier, et al. (2002) Proc. Natl. Acad. Sci. USA 99:13079-84). By biochemical techniques, it has been shown that anionic lipids, such as 1-palmitoryl-2-oleoylphosphatidylglycerol (POPG) and 1-palmitoryl-2-oleoylphosphatidylserine (POPS), bind to full-length recombinant PrP (rPrP) and induce the conformational cnange associated with PK-resistance (Wang (2008) Ph.D. Dissertation, The Ohio State University). Conversion of PrPC to PrPSc has also been shown with co-purified, uncharacterized lipid and poly(A) RNA molecules (Deleault, et al. (2007) Proc. Natl. Acad. Sci. USA 104:9741-6).

While PrP^{Sc} detection limits of 2 pM, corresponding to an aggregate concentration of approximately 2 fM (Bieschke, et al. (2000) Proc. Natl. Acad. Sci. USA 97(10):5468-73) to 50 pg PrP^{Sc} (Barnard, et al. (2000) Luminescence 15: 357-362), have been reported using immunoassays, improved methods of increasing the detection limits are needed to enhance the detection limits of these assays so that prion diseases may be detected at the earliest possible stages of development.

### SUMMARY OF THE INVENTION

The present invention features a method for facilitating propagation and production of PrP^{Sc} by contacting PrP^{C} with PrP^{Sc} in the presence of phosphatidylethanolamine or sphingomyelin.

The present invention also features a method for determining the presence of an infectious prion protein in a sample by contacting a sample suspected of containing an infectious prion protein with phosphatidylethanolamine or sphingomyelin, and detecting the presence of PrP^{Sc}, wherein the presence of PrP^{Sc} is indicative of an infectious prion protein in the sample. In one embodiment of this method, the sample is further contacted with exogenous PrP^{C}.

A method for identifying a PrP effector is also provided. This method of the invention involves the steps of

(i) contacting a sample containing PrP^{Sc} with phosphatidylethanolamine or sphingomyelin in the presence and absence of a test compound;

(ii) contacting the sample with a PrP^{C}; and

(iii) determining whether the test compound is a PrP effector. PrP effectors are also provided, as are pharmaceutical compositions and compositions for disinfecting prion-contaminated substances.

In accordance with some embodiments of these methods of the invention, the PrP^{C} is recombinant PrP^{C}. In accordance with other embodiments, the phosphatidylethanolamine or sphingomyelin is purified. In specific embodiments, the phosphatidylethanolamine is listed in Table 1.

A kit for facilitating propagation and production of PrP^{Sc} is also embraced by this invention. Such a kit includes PrP^{C} and one or more purified phosphatidylethanolamine or sphingomyelin, wherein in some embodiments the PrP^{C} is recombinant and in other embodiments the phosphatidylethanolamine is one or more of the phosphatidylethanolamines in Table 1.

### BRIEF DESCRIPTION OF THE DRAWINGS

FIG. 1 shows the effect of purified brain phosphatidylethanolamine (Brain PE) and sphingomyelin on PrP^{Sc} amplification and propagation. Shown are western blots of 3-round serial propagation reactions probed with anti-PrP antibody 6D11. For each series, a sample of 50% input recombinant mouse PrP not subject to protease digestion (—PK) is shown for comparison, followed by four protease-digested samples representing rounds 0 (input PrP^{Sc}), 1, 2, and 3 (amplified and propagated PrP^{Sc}). No lipid cofactor was added to the control series on the left, and Drain PE or sphingomyelin was added to the series on the right.

FIG. 2 shows the effect of various synthetic PE molecules on PrP^{Sc} propagation. Shown are western blots of 3-round serial propagation reactions containing various synthetic PE molecules. Blots were probed with anti-PrP antibody 6D11. A sample of 50% input recombinant mouse PrP not subject to protease digestion (—PK) is shown for comparison, followed by three protease-digested samples representing rounds 1, 2, and 3 (propagated PrP^{Sc}).

FIG. 3 shows the effect of various synthetic PE plasmaslogen molecules on PrP^{Sc} propagation. Shown are western blots of 3-round serial propagation reactions containing various synthetic PE plasmalogen molecules, as indicated. Blots were probed with anti-PrP antibody 6D11. A sample of 50% input recombinant mouse PrP not subject to protease digestion (—PK) is shown for comparison, followed by 3 protease-digested samples representing rounds 1, 2, and 3 (propagated PrP^{Sc}).

### DETAILED DESCRIPTION OF THE INVENTION

Infectious prions have now been successfully amplified in vitro using phosphatidylethanolamine and sphingomyelin, which potently and efficiently permit prion amplification. Prion amplification facilitated by lipids can occur either with continuous shaking or intermittent sonication, and can be seeded by small amounts of prion-infected tissue. Having identified phosphatidylethanolamine and sphingomyelin as endogenous prion conversion cofactors, this invention finds application in the detection of infectious prions, the generation of large amounts of recombinant infectious prions for research and diagnostic purposes, and in the identification of PrP effectors for use in the prevention or treatment of a prion disease or in the disinfection of prion-contaminated substances.

For the purposes of the present invention, “PrP^{C}” is the common or cellular prion protein that is widely expressed within the body of mammals. The structure of PrP^{C} is highly conserved and is not associated with a disease state. For use in the methods of the present invention, PrP^{C} can be isolated and optionally purified (e.g., to greater than 80%, 90%, 95%, or 99% homogeneity) from a natural source (e.g., brain tissue) or produced by recombinant technology. Production of recombinant PrP^{C} is routinely practiced in the art and described, for example, by Geoghegan, et al. (2009) PLoS Pathog. 5:e1000535; Makarava & Baskakov (2008) Methods Mol. Biol. 459:131-43; and Dan, et al. (2009) J. Biomol. Tech. 20:241-8. Furthermore, the PrP^{C} can be tagged to facilitate purification. See, e.g., Coleman, et al. (2009) Biochem. Biophys. Res. Commun. 380:564-568. In particular embodiments, the PrP^{C} of the methods and kit of the invention is recombinant PrP^{C}.

Infectious prions are composed of a modified form of the PrP^{C} protein, and are called “PrP^{Sc}”. In this respect, the term “PrP^{Sc}” refers to the conformationally altered form of the PrP^{C} molecule that has been associated with diseases such as TSE/prion diseases, including vCJD, CJD, kuru, fatal insomnia, GSS, scrapie, BSE, CWD, and other TSEs, including rare TSEs of captive and experimental animals. PrP^{Sc} has the same primary amino acid sequence as PrP^{C}, but due to a post-translational conformational change, α-helices are transformed into β-sheets. PrP^{C} contains three α-helices and has little β-sheet structure; in contrast, PrP^{Sc} is rich in β-sheet structure. Whereas PrP^{C} is highly sensitive to proteinase K, PrP^{Sc} can be partially digested to form PrP^{Res}. In this respect, the term “PrP^{Res}” refers to the proteinase resistant derivatives of the PrP^{Sc} protein of molecular weight 27-30 kDa, which remain following partial digestion of PrP^{Sc} with proteinase K. In this respect, PrP^{Res} may also be referred to as “PrP27-30.” Accordingly, in the context of the present invention, the methods disclosed herein can be carried out with either PrP^{Sc} or PrP^{Res}. PrP^{Sc} can be isolated and optionally purified from a natural source (e.g., brain tissue) or obtained via in vitro amplification of PrP^{C}, wherein subsequent protease digestion yields PrP^{Res}.

As the results herein demonstrate, lipids such as phosphatidylethanolamine and sphingomyelin facilitate the propagation of PrP^{Sc} from PrP^{C}, when compared to control samples lacking these lipids. Propagation of PrP^{Sc} is enhanced by phosphatidylethanolamine or sphingomyelin by increasing the rate and/or amount of PrP^{C} that is converted to PrP^{Sc}. In this respect, greater than 80%, 90%, 95%, or 99% of the input substrate is converted to PrP^{Sc} during a conventional amplification reaction (e.g., incubation at 37° C. for either 1-3 days with intermittent or continuous shaking, or intermittent sonication). Accordingly, the present invention embraces a method for facilitating the propagation and production of PrP^{Sc} by contacting PrP^{C} with PrP^{Sc} in the presence of phosphatidylethanolamine or sphingomyelin. Using this method, large amounts of PrP^{Sc} and/or PrP^{Res} can be produced. The PrP^{Sc} and/or PrP^{Res} so produced can be used in research, e.g., in determining the crystal structure of PrP^{Sc} and/or PrP^{Res} or the interaction sites of various proteins and chemical compounds to prions; in the diagnosis of prion diseases (e.g., as seed in amplification reactions or as a standard or control); and/or in screening assays to identify agents that inhibit conversion or block activity of infectious prion protein. Moreover, when each reactant is used in substantially purified form, the resulting product will be substantially homogenous to PrP^{Sc} and/or PrP^{Res} and lipid cofactor.

As with PrP^{Sc} and PrP^{C}, phosphatidylethanolamines or sphingomyelins of use in this invention can be isolated and optionally purified from a natural source, or chemically synthesized or purchased from a commercial source in a substantially purified form. Independent of the method for obtaining purified phosphatidylethanolamine or sphingomyelin, particular embodiments of this invention embrace phosphatidylethanolamine or sphingomyelin of greater than 80%, 90%, 95%, or 99% homogeneity. When used in the kits and methods of this invention, the phosphatidylethanolamine or sphingomyelin may consist of one particular type of phosphatidylethanolamine or sphingomyelin. Alternatively, mixtures of purified phosphatidylethanolamines or purified sphingomyelins (i.e., one or more types of phosphatidylethanolamines or sphingomyelins) may be employed. Moreover, mixtures of purified phosphatidylethanolamines and sphingomyelins may be used.

As is known in the art, phosphatidylethanolamine (PE) is a lipid having the following structure:

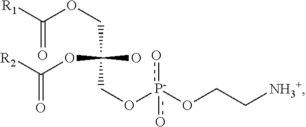

wherein R₁ and R₂ represent acyl chains of the same or different length, of the same or different degree of saturation, and same or different placement of double bonds. Moreover, derivatives of PE are also included, wherein one of the two acyl chains are attached via an ether alkenyl linkage (i.e., plasmologen) and/or are modified with, e.g., a label or tag including fluorescent labels such as NBD. Phosphatidylethanolamines of particular use in accordance with the present invention have acyl chain lengths of from 12C to 22C with 0 to 6 double bonds. While certain phosphatidylethanolamines were more active than others in the assay conditions exemplified herein, it is expected that the activity of these latter phosphatidylethanolamines may be increased by modifying the assay conditions, e.g., by changing the concentration and/or type of the detergent employed. In certain embodiments, the kits and methods of this invention employ one or more of the phosphatidylethanolamine types listed in Table 1.

TABLE 1

| | Chemical Structure |
|---|---|
| Abbreviation | (Chemical Name) |
| 16:0-18:1 PE | 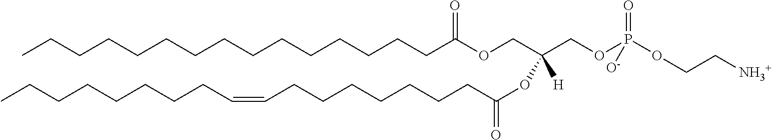 |
| | (1-palmitoyl-2-oleoyl-sn-glycero-3-phosphoethanolamine) |
| 16:0-22:6 PE | 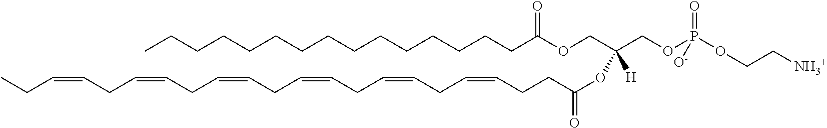 |
| | (1-palmitoyl-2-docosahexaenoyl-sn-glycero-3-phosphoethanolamine) |
| 16:0-12:0 NBD PE | 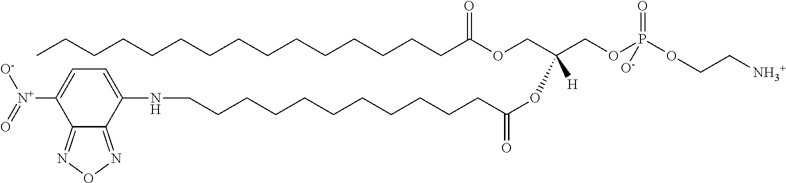 |
| | (1-palmitoyl-2-{12-[(7-nitro-2-1,3-benzoxadiazol-4-yl)amino]lauroyl}-sn-glycero-3-phosphoethanolamine) |
| C18(Plasm)- 18:1 PE | 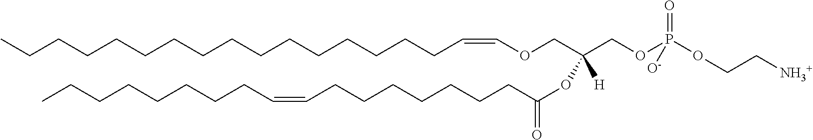 |
| | (1-(1Z-octadecenyl)-2-oleoyl-sn-glycero-3-phosphoethanolamine) |
| C18(Plasm)- 20:4 PE | 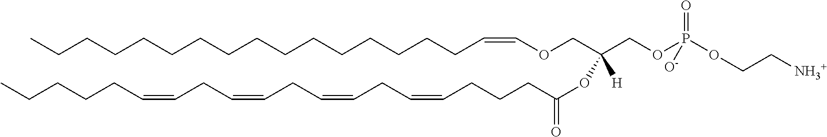 |
| | (1-(1Z-octadecenyl)-2-arachidonoyl-sn-glycero-3-phosphoethanolamine) |
| C18(Plasm)- 22:6 PE | 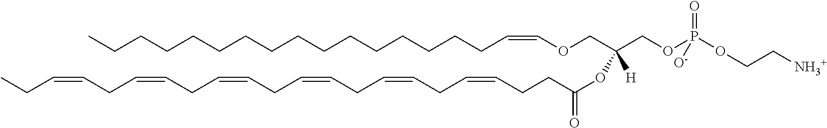 |
| | (1-(1Z-octadecenyl)-2-docosahexaenoyl-sn-glycero-3-phosphoethanolamine) |

Sphingomyelin is also known in the art as a sphingolipid characterized as a ceramide bearing either a phosphocholine or a phosphatidylethanolamine moiety as a head group. Although sphingomyelins differ chemically from phosphatidylcholine and phosphatidylethanolamine, the conformation and charge distribution are similar. Accordingly, while one embodiment features a sphingomyelin with a phosphatidylcholine head group, in particular embodiments, a sphingomyelin of use in the instant methods and kits contains a phosphatidylethanolamine head group. A sphingomyelin of the invention can have an acyl chain length of from 12C to 22C with 0 to 6 double bonds. Moreover, derivatives of sphingomyelin are also included, wherein acyl chains are modified with, e.g., a label or tag including fluorescent labels such as NBD.

For convenient use in the methods of this invention, PrP^{C} and purified phosphatidylethanolamine(s) and/or sphingomyelin(s) are provided in the form in a kit. A kit of the invention typically includes a container holding one or more phosphatidylethanolamines and/or sphingomyelins, a container holding PrP^{C}, and instructions for using the PrP^{C} and phosphatidylethanolamine(s) and/or sphingomyelin(s) for the purpose of amplifying PrP^{Sc}. Examples of containers include multi-well plates which allow simultaneous identification of PrP^{Sc} in multiple samples. The kit can further contain a heterocyclic compound such as imidazole for binding inhibitory divalent metals and/or other reagents which increase, enhance or stimulate the amplification of PrP^{Sc}. For example, the phosphatidylethanolamine(s) and/or sphingomyelin(s) can be combined with detergents such as TRITON® (X-100 or X-114), TWEEN® (e.g., 20 or 80), BRIJ®, GENAPOL®, CHAPS, CHAPSO ZWITTERGENT® (e.g., 3-16, 3-14, 3-12, 3-10, or 3-8), THESIT®, sarkosyl, deoxycholate (e.g., sodium deoxycholate, sodium taurodeoxycholate, or sodium glycodeoxycholate), NP-40, sodium dodecyl sulfate, digitonin, or cetyltrimethylammonium bromide (CTAB); salts such as KCl, MgCl₂, or NaCl; buffers such as phosphate-buffered saline, tris-buffered saline, MOPS, HEPES, PIPES, Glycylglycine, MES or citrate buffer; chelating agents such as EDTA or EGTA; or other natural cofactors.

Propagation of PrP^{Sc} from PrP^{C} in the presence of phosphatidylethanolamine and/or sphingomyelin can be carried out under any suitable conditions conventionally used in the conversion or amplification of PrP^{Sc}. Such reaction conditions include those described herein and those used in the in vitro PrP^{Sc} amplification method described by Lucassen, et al. ((2003) supra) and PMCA method of Saborio, et al. ((2001) supra). To facilitate the propagation and production of PrP^{Sc} from PrP^{C}, desirably the phosphatidylethanolamine or sphingomyelin is used in a concentration ranging from 0.001 mM to 10 mM.

In light of the detection limits of conventional assays, efficient propagation of PrP^{Sc} from PrP^{C} can enhance the sensitivity of assays for detecting and diagnosing TSE/prion diseases. In this respect, the present invention also embraces a method for determining the presence of an infectious prion protein in a sample by contacting a sample suspected of containing an infectious prion protein with phosphatidylethanolamine and/or sphingomyelin so that the PrP^{Sc} present in the sample is amplified or propogated. Amplified PrP^{Sc} can then be detected using any conventional approach including, but not limited to SDS-PAGE, antibody-based detection, and the like. If PrP^{Sc} (including PrP^{Res}) is detected, it can be concluded that the sample used in the assay contains an infectious prion protein. For samples with little or no endogenous PrP^{C}, e.g., purified protein samples, food samples or environmental samples, particular embodiments of the present method include adding exogenous PrP^{C} (e.g., isolated and optionally purified or recombinant PrP^{C}) to the sample.

The term “sample” is used herein to denote any solution, suspension, extract, composition, preparation, product, component, tissue, organ, cell, or other entity having or suspected of having an infectious prion protein. Samples according to certain aspects and embodiments of the present invention include, but are not limited to, biological samples, food products, environmental samples, or water samples. Biological samples include, but are not limited to, blood-derived samples; brain-derived samples; bodily fluids, such as, but not limited to, blood, plasma, serum, cerebrospinal fluid, urine, saliva, milk, ductal fluid, tears, or semen; biological extracts, such as collagen extracts, gland extracts, or tissue homogenates or extracts. Biological samples are derived from humans or animals, including but not limited to bovine, ovine, porcine, equine, murine, or Cervidae animals. Blood-derived samples include, but are not limited to, platelet concentrates, plasma protein preparations, immunoglobulin preparations, fibrinogen preparations, factor XIII preparations, thrombin preparations, factor VIII preparations, von Willebrand factor preparations, protein C preparations, or activated protein C preparation. The samples according to certain aspects and embodiments of the present invention also include, but are not limited to, pharmaceutical compositions, therapeutic compositions, a cosmetic compositions and products, food or food products, or nutritional supplement compositions. The examples of food-product samples include, but are not limited to, gelatin, jelly, milk, dairy products, collagen, or an infant formula.

The samples, according to certain aspects, include protein solutions containing various proteins, including, but not limited to, human or animal serum albumin. For example, the samples include, but are not limited to, therapeutic products containing human serum albumin; human or animal serum albumin preparations; or preparations containing human or animal serum albumin as a stabilizer.

Environmental samples include, but are not limited to, soil, sewage or water, such as water from a source such as a stream, river, aquifer, well, water treatment facility or recreational water.

Other samples include, but are not limited to, liquid samples, solid samples, or colloidal samples. A solid sample can be extracted with an aqueous solvent, an organic solvent or a critical fluid, and the resulting supernatant can be contacted with the binding materials. Examples of solid samples include, but are not limited to, animal-derived products, particularly those that have been exposed to agents that transmit prions, e.g., bone meal-derived from bovine sources, brain tissue, corneal tissue, fecal matter, bone meal, beef by-products, sheep, sheep by-products, deer and elk, deer and elk by-products, and other animals and animal-derived products.

As the results herein demonstrate, phosphatidylethanolamine and sphingomyelin facilitate the propagation of PrP^{Sc} from PrP^{C}. Accordingly, compounds that modulate the interaction between a PrP and phosphatidylethanolamine or sphingomyelin could, depending on the nature of the compound, enhance, stimulate, delay or block the conversion of PrP^{C} to PrP^{Sc}. Thus, phosphatidylethanolamine and/or sphingomyelin can be used in binding studies or conversion assays with PrP to screen for agents that block, promote or disrupt the PrP-phosphatidylethanolamine or PrP-sphingomyelin interaction. In this respect, the present invention also embraces a method for identifying a PrP effector by (i) contacting a sample containing PrP^{Sc} with phosphatidylethanolamine and/or sphingomyelin in the presence and absence of a test compound; (ii) contacting the sample with a PrP^{C}; and (iii) determining whether the test compound is a PrP effector.

A PrP effector is defined as a compound that modulates (i.e., increases or decreases) the amount or rate of PrP^{Sc} produced as compared to the amount or rate of PrP^{Sc} produced in the absence of the compound; blocks binding of phosphatidylethanolamine or sphingomyelin to PrP^{C}; disrupts the interaction between PrP^{Sc} and phosphatidylethanolamine or sphingomyelin; or binds to a PrP^{Sc}/phosphatidylethanolamine or PrP^{Sc}/sphingomyelin complex. In this respect, PrP^{C} can be added to the sample before or at the same time the sample is contacted with phosphatidylethanolamine or sphingomyelin in order to identify agents that modulate the interaction between PrP^{C} and phosphatidylethanolamine or sphingomyelin. Alternatively, PrP^{C} can be added to the sample after the sample is contacted with phosphatidylethanolamine or sphingomyelin in order to identify agents that disrupt or bind to a PrP^{Sc}/phosphatidylethanolamine or PrP^{Sc}/sphingomyelin complex. An example of a readout for the assay of the invention can employ an antibody specific for PrP^{Sc}, wherein increases in the amount or rate of PrP^{Sc} production is indicative of a stimulatory agent, whereas decreases in the amount or rate of PrP^{Sc} production is indicative of an inhibitory agent.

Effectors can be identified by screening a library of test compounds. A library can include either collections of pure compounds or collections of compounds mixtures. Examples of pure compounds include, but are not limited to, metal ions, proteins, polypeptides, peptides, nucleic acids, oligonucleotides, carbohydrates, lipids, synthetic or semi-synthetic chemicals, and purified natural products. Examples of compounds mixtures include, but are not limited to, extracts of prokaryotic or eukaryotic cells and tissues, as well as fermentation broths and cell or tissue culture supernates. In the case of compounds mixtures, one may not only identify those crude mixtures that possess the desired activity, but also monitor purification of the active compound from the mixture for characterization and development as a therapeutic drug. In particular, the mixture so identified can be sequentially fractionated by methods commonly known to those skilled in the art which include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active compound is obtained.

Library screening can be performed in any format that allows rapid preparation and processing of multiple reactions such as in, for example, multi-well plates of the 96-well variety. Stock solutions of the compounds as well as assay components are prepared manually and all subsequent pipetting, diluting, mixing, washing, incubating, sample readout and data collecting is done using commercially available robotic pipetting equipment, automated work stations, and analytical instruments for detecting the signal generated by the assay. Examples of such detectors include, but are not limited to, luminometers, spectrophotometers, calorimeters, and fluorimeters, and devices that measure the decay of radioisotopes.

PrP effectors identified by the screening assay of this invention have various uses including the prevention or treatment of a TSE/prion disease as well as in the detection, removal, or inactivation of infectious prions. For example, an effector that increases, enhances or stimulates the interaction between PrP^{C} and phosphatidylethanolamine or sphingomyelin, or the amount or rate of formation of PrP^{Sc} produced, is useful in methods for enhancing the sensitivity of detecting PrP^{Sc}, or for facilitating the production of PrP^{Sc}. An effector that decreases, inhibits, blocks or disrupts the interaction between PrP^{C} and phosphatidylethanolamine or sphingomyelin, or the amount or rate of PrP^{Sc} formation, is useful for preventing or treating a TSE/prion disease. Accordingly, the present invention also embraces a pharmaceutical composition containing a PrP effector in admixture with a pharmaceutically acceptable carrier (e.g., saline, sugar, starch, and the like) for use in the prevention or treatment of a TSE/prion disease. Moreover, the invention also embraces a composition containing a PrP effector in admixture with a suitable carrier (e.g., water, alcohol, NaOH or NaOCl) for use in the disinfection of prion-contaminated substances (e.g., samples or surfaces). By way of illustration, it is contemplated that agents such as duramycin, which bind phosphatidylethanolamine (Zhao, et al. (2008) J. Nucl. Med. 49:1345-520), can have the dual role of exerting antibiotic activity and disinfecting prion-contaminated substances.

The invention is described in greater detail by the following non-limiting examples.

### Example 1

### In Vitro Conversion Assay Method

Samples of 100 μl total volume containing 6 μg/ml recombinant mouse PrP expressed in E. coli, 20 mM Tris pH 7.5, 135 mM NaCl, 5 mM EDTA, 0.3%-1.0% TRITON X-100, and varying concentrations of purified or synthetic lipids (purchased from Avanti Polar Lipids, Alabaster, Ala.) were seeded with 0.6 μg/ml PrP^{Sc} and incubated at 37° C. for 1-3 days with intermittent or continuous shaking to complete the first round of PrP^{Sc} amplification. Following each round of amplification, 10 μl of each sample was removed and used to seed a new 100 μl reaction cocktail containing all components listed above, with the exception of PrP^{Sc}. A 30 μl aliquot from each round was subjected to digestion with proteinase K, and analyzed by western blot with monoclonal antibody 6D11 to measure PrP^{Sc} levels. In each blot, a 15 μl aliquot not subjected to proteinase K digestion (—PK) was used for comparison.

### Example 2

### Phosphatidylethanolamine-Mediated Conversion or Recombinant PrP

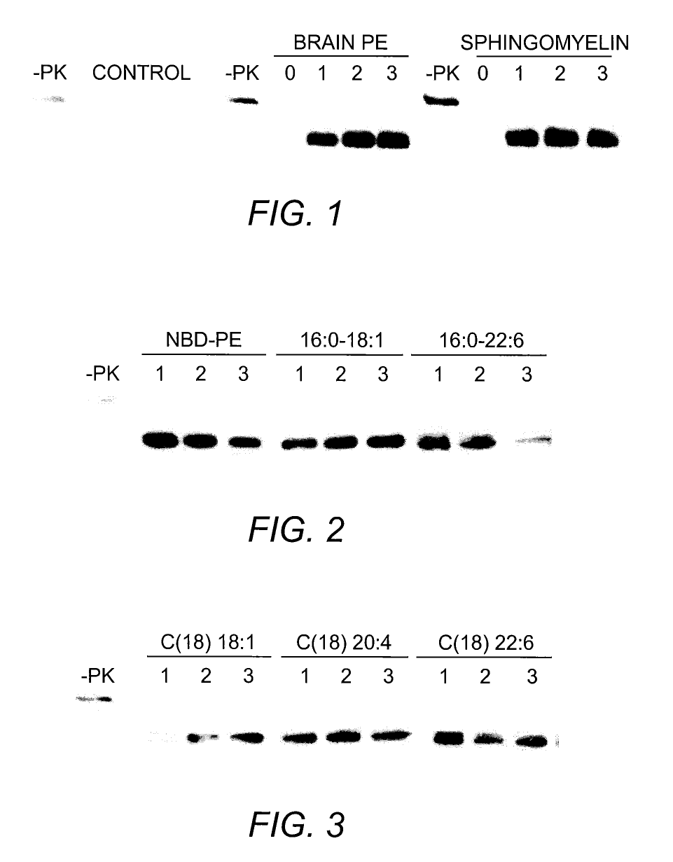

Addition of brain-derived, purified phosphatidylethanolamine (PE) (containing 50% plasmalogen and a mixture of diacyl forms) successfully facilitated the amplification and propagation of PrP^{Sc}, compared to control reactions lacking lipid cofactor (FIG. 1). Nearly 100% of the input recombinant PrP substrate was converted into PrP^{Sc}. Addition of brain-derived, purified sphingomyelin also facilitated the conversion of recombinant PrP (FIG. 1). In contrast, phosphatidylcholine, phosphatidylinositol, phosphatidylserine, phosphatidic acid, sulfatides, cerebrosides, and phosphatidylglycerol facilitate prion amplification to a lesser extent.

To determine the optimal acyl group composition of the PE cofactor, various synthetic PE molecules were tested for their ability to facilitate PrP^{Sc} propagation. The results showed that synthetic 16:0-18:1 PE, 16:0-22:6 PE, 16:0-12:0 NBD PE, C18(Plasm)-18:1 PE, C18(Plasm)-20:4 PE, and C18(Plasm)-22:6 PE all successfully facilitated propagation (FIGS. 2 and 3), whereas 16:0-20:4 PE, 12:0-12:0 PE, 18:0-18:0 PE, and 18:0-18:1 PE were unable to facilitate propagation of PrP^{Sc} under the same assay conditions.

1. A method for facilitating propagation and production of PrP^{Sc} comprising contacting PrP^{C} with PrP^{Sc} in the presence of phosphatidylethanolamine or sphingomyelin so that propagation and production of PrP^{Sc} from PrP^{C} is facilitated.

2. The method of claim 1, wherein the PrP^{C} is recombinant PrP^{C}.

3. The method of claim 1, wherein the phosphatidylethanolamine or sphingomyelin is purified.

4. The method of claim 1, wherein the phosphatidylethanolamine comprises one or more of the phosphatidylethanolamines in Table 1.

5. A method for determining the presence of an infectious prion protein in a sample comprising contacting a sample suspected of containing an infectious prion protein with phosphatidylethanolamine or sphingomyelin, and detecting the presence of PrP^{Sc}, wherein the presence of PrP^{Sc} is indicative of an infectious prion protein in the sample.

6. The method of claim 5, wherein the step of contacting the sample is carried out in the presence of exogenous PrP^{C}.

7. The method of claim 6, wherein the exogenous PrP^{C} is recombinant.

8. The method of claim 5, wherein the phosphatidylethanolamine or sphingomyelin is purified.

9. The method of claim 5, wherein the phosphatidylethanolamine comprises one or more of the phosphatidylethanolamines in Table 1.

10. A method for identifying a PrP effector comprising:
(i) contacting a sample containing PrP^{Sc} with phosphatidylethanolamine or sphingomyelin in the presence and absence of a test compound;
(ii) contacting the sample with a PrP^{C}; and
(iii) determining whether the test compound is a PrP effector.

11. The method of claim 10, wherein the wherein the PrP^{C} is recombinant.

12. The method of claim 10, wherein the phosphatidylethanolamine or sphingomyelin is purified.

13. The method of claim 10, wherein the phosphatidylethanolamine comprises one or more of the phosphatidylethanolamines in Table 1.

14. The method of claim 10, wherein the step of determining whether the compound is a PrP effector comprises determining whether the test compound modulates the amount of PrP^{Sc} produced in the presence of said test compound as compared to the absence of said test compound.

15. A PrP effector identified by the method of claim 10.

16. A pharmaceutical composition for the prevention or treatment of a TSE/prion disease comprising a PrP effector of claim 15 in admixture with a pharmaceutically acceptable carrier.

17. A composition for disinfection of prion-contaminated substances comprising a PrP effector of claim 15 in admixture with a suitable carrier.

18. A kit for facilitating the propagation and production of PrP^{Sc} comprising PrP^{C} and one or more purified phosphatidylethanolamine, or one or more purified sphingomyelin.

19. The kit of claim 18, wherein the PrP^{C} is recombinant.

20. The kit of claim 18, wherein the phosphatidylethanolamine comprises one or more of the phosphatidylethanolamines in Table 1.
